(19) United States
(12) Patent Application Publication
(10) Pub. No.: US 2023/0001035 A1
(43) Pub. Date: Jan. 5, 2023

(54) MOVABLE CURTAIN DISINFECTION DEVICE

(71) Applicant: INDUSTRY-UNIVESITY COOPERATION FOUNDATION HANYANG UNIVERSITY, Seoul (KR)

(72) Inventors: Yeong Tak SONG, Seoul (KR); Tae Ho LIM, Seoul (KR); Jung Hyun KIM, Seoul (KR); Min Hyuk LEE, Seoul (KR); Jong Bong CHOI, Seoul (KR); Hyun Ji SUNG, Seoul (KR); Kyeng Jin LIM, Seoul (KR)

(73) Assignee: INDUSTRY-UNIVESITY COOPERATION FOUNDATION HANYANG UNIVERSITY, Seoul (KR)

(21) Appl. No.: 17/776,875

(22) PCT Filed: Nov. 10, 2020
(86) PCT No.: PCT/KR2020/014608

§ 371 Date: May 13, 2022

(30) Foreign Application Priority Data
Nov. 20, 2019 (KR) 10-2019-0149860

(51) Int. Cl. A61L 2/26; B65H 20/02; A61L 2/14; A61L 2/10; A61L 2/04; A61L 2/18

(52) U.S. Cl. CPC A61L 2/26; B65H 20/02; A61L 2/14; A61L 2/10; A61L 2/04; A61L 2/18; B65H 2404/144; B65H 2402/41; A61L 2202/26; A61L 2202/11

(57) ABSTRACT

A movable curtain disinfection device according to an embodiment of the present invention includes frames installed on an upper portion of a support, spaced apart from each other to face each other, and configured to define a disinfection space for a curtain provided between the frames, rotary rollers provided so that a pair of rotary rollers thereof is mounted on an inner surface of each of the frames, the rotary rollers being configured to rotate so that the curtain passes through the disinfection space, rotary belts each configured to surround an outer portion of each of the pairs of rotary rollers, the rotary belts being configured to guide a movement of the curtain while rotating in conjunction with rotations of the rotary rollers, and disinfection modules disposed between the pairs of rotary rollers and configured to disinfect the curtain passing through the disinfection space.

### TECHNICAL FIELD

The present invention relates to a movable curtain disinfection device, and more particularly, to a movable curtain disinfection device using a technology related to plasma, heat, ultraviolet rays, disinfectants, and the like.

### BACKGROUND ART

Recently, as viral diseases such as severe acute respiratory syndrome (SARS) and novel influenza occur and circulate, and the perception of disinfection is increasing. In particular, there are social issues on cross-infection between infected patients in hospitals and infection caused by the spread of virus.

In particular, the spread of disease was amplified because of cross-infection between patients due to the Middle East Respiratory Syndrome (MERS) in 2015, resulting in significant social confusion and loss. As a result, there has been increased social awareness of the importance of spatial disinfection and management of spatial disinfection in hospitals.

However, there is a limitation in disinfection in the hospital despite the growth of awareness of infection management. While guidelines on managing high-infectious medical devices are established, methods of cleaning walls, floors, or the like are not specified. In particular, a method of cleaning a curtain used to protect the patient's privacy is performed just by immediately replacing a dirty or contaminated curtain with a washed curtain, and each specified department merely conforms to the cycle of replacing curtains.

In general, if patients cannot be isolated, curtains are placed between the patients and ensure distances of at least 1 m between the patients and other patients or visitors. Therefore, the function of the curtain is also used to block infection. In addition, because many people touch the curtain with their hands several tens of times a day, it is very important to manage infection through the curtain.

As a related art, there is Korean Utility Model Registration No. 20-0418452 (entitled Ultraviolet Sterilization Device for Mobile Device and published on Jun. 12, 2006).

### DISCLOSURE

### Technical Problem

An embodiment of the present invention provides a movable curtain disinfection device including movable frames having a disinfection module using plasma, heat, ultraviolet ray, disinfectants, and the like, thereby preventing infectious diseases by eliminating bacteria and viruses attached to a curtain in a hospital room.

Technical problems to be solved by the present invention are not limited to the above-mentioned technical problem(s), and other technical problem(s), which are not mentioned above, may be clearly understood by those skilled in the art from the following descriptions.

### Technical Solution

An embodiment of the present invention provides a movable curtain disinfection device including: frames installed on an upper portion of a support, spaced apart from each other to face each other, and configured to define a disinfection space for a curtain provided between the frames; rotary rollers provided so that a pair of rotary rollers thereof is mounted on an inner surface of each of the frames, the rotary rollers being configured to rotate so that the curtain passes through the disinfection space; rotary belts each configured to surround an outer portion of each of the pairs of rotary rollers, the rotary belts being configured to guide a movement of the curtain while rotating in conjunction with rotations of the rotary rollers; and disinfection modules disposed between the pairs of rotary rollers and configured to disinfect the curtain passing through the disinfection space.

In addition, the rotary rollers according to the embodiment of the present invention may include two pairs of rotary rollers mounted on the inner surfaces of the frames so as to face one another, and the rotary rollers may be mounted in a longitudinal direction of the frame and disposed at two opposite sides of the inner surface of the frame.

In addition, the movable curtain disinfection device according to the embodiment of the present invention may further include elastic members each disposed rearward of each of the pairs of rotary rollers and configured to press the rotary rollers in a direction in which the rotary rollers face one another so that the curtain passes through the disinfection space while being supported by the rotary rollers.

In addition, the rotary belts according to the embodiment of the present invention may be provided at upper and lower sides of the rotary rollers.

In addition, the disinfection space according to the embodiment of the present invention may be sealed as the rotary belts are in surface contact with the curtain passing through the disinfection space.

In addition, the disinfection module according to the embodiment of the present invention may be a module detachably mounted on the inner surface of the frame and include at least one of a plasma-based disinfector, a heat-based disinfector, an ultraviolet-based disinfector, and a liquid-disinfectant-based disinfector.

In addition, the liquid-disinfectant-based disinfector, the ultraviolet-based disinfector, the plasma-based disinfector, and the heat-based disinfector of the disinfection module according to the embodiment of the present invention may be sequentially disposed when the curtain is made of woven fabric, and the liquid-disinfectant-based disinfector, the ultraviolet-based disinfector, and the plasma-based disinfector may be sequentially disposed when the curtain is made of synthetic resin.

In addition, the movable curtain disinfection device according to the embodiment of the present invention may further include a plurality of wheels disposed on a lower portion of the support and configured to move the frame while supporting the frame.

In addition, the movable curtain disinfection device according to the embodiment of the present invention may further include a height adjustment member provided to be embedded in the support, connected between the lower portion of the support and the wheels, and configured to adjust a height in a vertical direction.

In addition, the movable curtain disinfection device according to the embodiment of the present invention may further include an operation panel disposed on an outer surface of the frame and configured such that operation commands related to operations of the rotary roller, the rotary belt, and the disinfection module are inputted into the operation panel.

Other detailed matters of the embodiment are included in the detailed description and the accompanying drawings.

### Advantageous Effects

According to the embodiments of the present invention, the movable frames having the disinfection module using plasma, heat, ultraviolet ray, disinfectants, and the like may be implemented, which makes it possible to prevent infectious diseases by eliminating bacteria and viruses attached to the curtain in the hospital room.

In addition, according to the embodiments of the present invention, since the curtain disinfection device has the wheels, it is possible to reduce the inconvenience of having to separately collect and wash the curtains.

### DESCRIPTION OF DRAWINGS

FIG. 1 is a perspective view for explaining a movable curtain disinfection device according to an embodiment of the present invention.

FIG. 2 is a perspective view for explaining an internal structure of FIG. 1.

FIGS. 3A and 3B are views for explaining various structures of frames according to the embodiment of the present invention.

FIG. 4A is a top plan view when the frames illustrated in FIG. 1 are viewed from above.

FIG. 4B is a transverse cross-sectional view of the frames illustrated in FIG. 1.

FIG. 5 is a view for explaining a height adjustment member according to the embodiment of the present invention.

FIG. 6 is a view illustrating an example in which a curtain is disinfected according to the embodiment of the present invention.

### DESCRIPTION OF MAIN REFERENCE NUMERALS OF DRAWINGS

1: Movable curtain disinfection device
10: Support
20: Curtain
110: Frame
120: Rotary roller
125: Elastic member
130: Rotary belt
140: Disinfection module
142: Liquid-disinfectant-based disinfector
144: Ultraviolet-based disinfector
146: Heat-based disinfector
148: Plasma-based disinfector
150: Wheel
160: Height adjustment member
170: Operation panel

### MODE FOR INVENTION

Advantages and/or features of the present invention and methods of achieving the advantages and features will be clear with reference to embodiments described in detail below together with the accompanying drawings. However, the present invention is not limited to the embodiments disclosed herein but will be implemented in various forms. The embodiments of the present invention are provided so that the present invention is completely disclosed, and a person with ordinary skill in the art to which the present invention pertains can fully understand the scope of the present invention. the present invention will be defined only by the scope of the appended claims. Like reference numerals indicate like constituent elements throughout the specification.

Hereinafter, exemplary embodiments of the present invention will be described in detail with reference to the accompanying drawings.

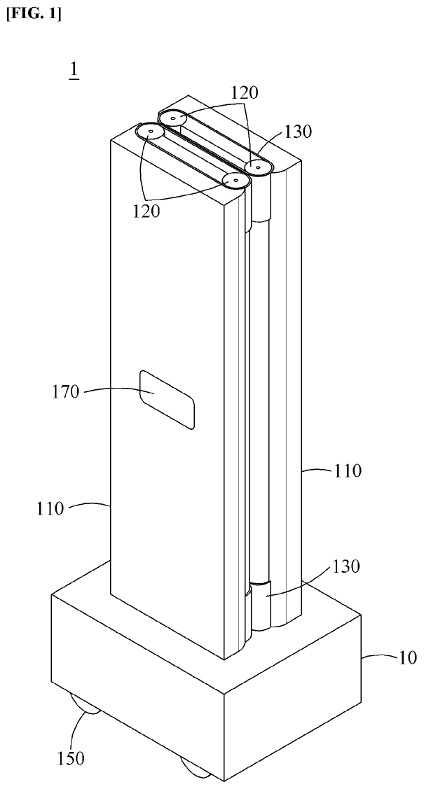

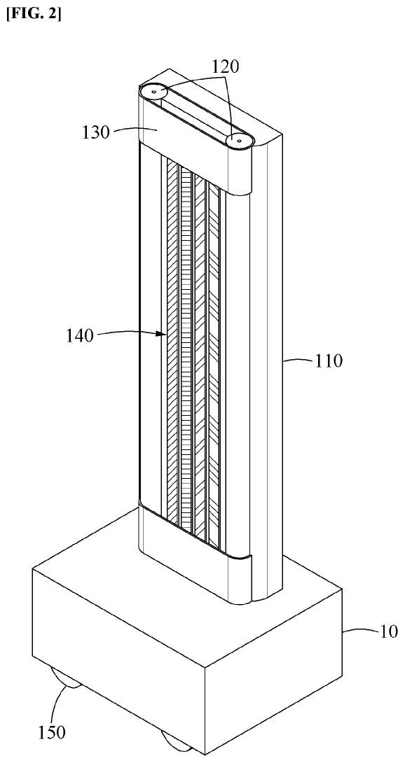

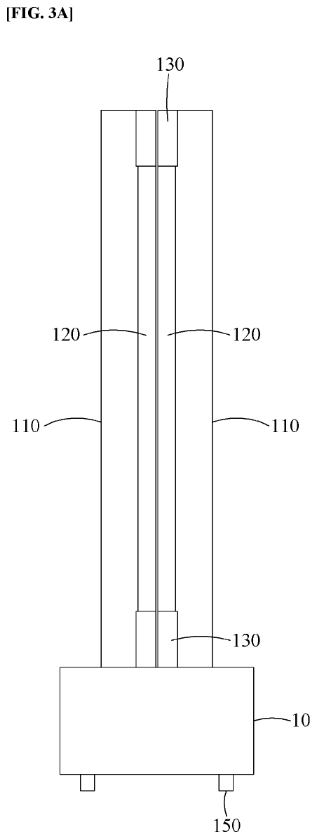

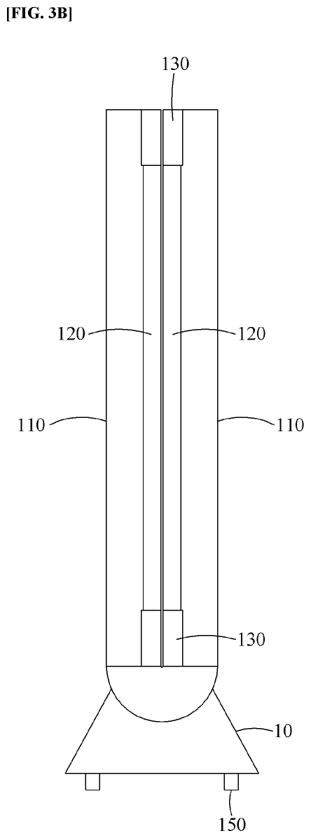

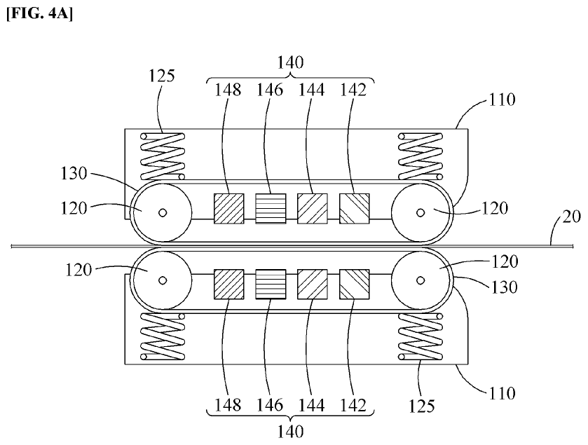

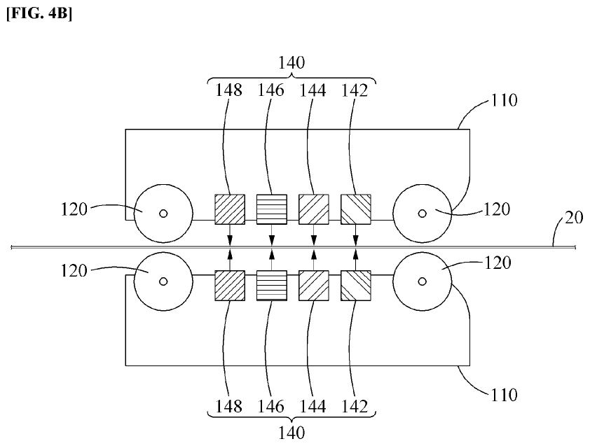

FIG. 1 is a perspective view for explaining a movable curtain disinfection device according to an embodiment of the present invention, FIG. 2 is a perspective view for explaining an internal structure of FIG. 1, FIGS. 3A and 3B are views for explaining various structures of frames according to the embodiment of the present invention, FIG. 4A is a top plan view when the frames illustrated in FIG. 1 are viewed from above, and FIG. 4B is a transverse cross-sectional view of the frames illustrated in FIG. 1.

Referring to FIGS. 1 and 2, a movable curtain disinfection device 1 according to an embodiment of the present invention may include a support 10, frames 110, rotary rollers 120, rotary belts 130, and disinfection modules 140.

The frames 110 may be disposed on the support 10 and spaced apart from one another to face each other, and a disinfection space for a curtain 20 may be provided between the frames 110 spaced apart from one another.

The frame 110 may be installed to have a predetermined length, and the predetermined length may generally correspond to a length of the curtain 20 commercially available.

The frames 110 may include a pair of frames 110 facing each other. The disinfection space may be provided in a fine gap defined as the pair of frames 110 is spaced apart from each other at a predetermined interval. For reference, the predetermined interval may have a narrow width that enables one sheet of the curtain 20 to pass therethrough.

The shape of the frame 110 may be variously implemented. For example, the frames may be disposed in a 11 shape as illustrated in FIG. 3A, or the frames may be disposed in a U shape as illustrated in FIG. 3B.

The rotary rollers 120 may be provided so that the pair of rotary rollers is mounted on an inner surface of each of the frames 110.

That is, among the pair of frames 110 facing each other, one pair of rotary rollers may be mounted on the inner surface of one frame 110, and the other pair of rotary rollers may be mounted on the inner surface of the other frame 110. In other words, the rotary rollers 120 may include two pairs of rotary rollers mounted on the inner surfaces of the frames 110 and facing one another.

The rotary rollers 120 may be mounted in a longitudinal direction of the frame 110 and disposed at two opposite sides of the inner surface of the frame 110. Therefore, the rotary rollers are disposed at a portion where the contaminated curtain 20 is inputted and a portion where the disinfected curtain 20 is outputted, such that the curtain 20 may pass through the disinfection space while being kept tense.

The rotary rollers 120 rotate so that the curtain 20 passes through the disinfection space.

To this end, the rotary roller 120 may have a cylindrical shape. However, the present invention is not limited thereto, and the rotary roller 120 may have any shape as long as the rotary roller 120 may rotate.

Meanwhile, as illustrated in FIG. 4A, the movable curtain disinfection device 1 according to the present invention may further include elastic members 125 each disposed rearward of each of the pairs of rotary rollers 120 and configured to press the rotary rollers 120 in a direction in which the rotary rollers 120 face one another.

The elastic members 125 may press the rotary rollers 120 toward the disinfection space so that the curtain 20 passes through the disinfection space while being supported by the rotary rollers 120.

Because the pressing force for pressing the rotary roller 120 is adjusted by an elastic force of the elastic member 125, all the curtains 20 having various thicknesses may be disinfected.

In the present embodiment, the elastic member 125 is provided in the form of a spring. However, the present invention is not limited thereto, and any elastic member may be used as long as the elastic member provides the elastic force.

The rotary belt 130 may surround an outer portion of each of the pairs of rotary rollers 120.

That is, the rotary belts 130 may be provided on one pair of rotary rollers and the other pair of rotary rollers. The rotary belt may surround the outer peripheries of the pair of rotary rollers while binding the pair of rotary rollers into one piece.

The rotary belts 130 may be disposed on upper and lower sides of the pair of rotary rollers 120, respectively.

The rotary belt 130 may guide the movement of the curtain 20 while rotating in conjunction with the rotation of the rotary rollers 120.

That is, the curtain 20, particularly, the upper and lower portions of the curtain 20 may move in one direction along the rotary belts 130 during a process in which the curtain 20 is inputted into the disinfection space, disinfected, and then outputted to the outside. In other words, the curtain 20 may be guided by the rotary belts 130 while passing through the disinfection space.

Therefore, the curtain 20 may pass through the disinfection space while being kept tense without being wrinkled or crumpled.

For reference, a rotation direction of the rotary roller 120 and a rotation direction of the rotary belt 130 may be equal to each other.

The disinfection space may be sealed as the rotary belts 130 are in surface contact with the curtain 20 passing through the disinfection space. That is, the two opposite surfaces of the upper and lower portions of the curtain 20 are completely in contact with the rotary belts 130, such that the contact between the disinfection space and the outside may be prevented. Therefore, the disinfection of the curtain 20 may be more effectively performed.

The disinfection modules 140 may be disposed between the pairs of rotary rollers.

That is, the disinfection modules 140 may be disposed between two rotary rollers constituting one pair of rotary rollers and two rotary rollers constituting the other pair of rotary rollers.

The disinfection module 140 is a module detachably mounted on the inner surface of the frame 110. The disinfection module 140 may be mounted on the frame 110 or separated from the frame 110, as necessary. Therefore, it is easy to dispose only the desired disinfection module 140 or replace the disinfection module 140 of which the lifespan is over.

The disinfection module 140 may include various disinfectors for disinfecting the curtain 20 passing through the disinfection space.

For example, the disinfection module 140 may include at least one of a plasma-based disinfector 148, a heat-based disinfector 146, an ultraviolet-based disinfector 144, and a liquid-disinfectant-based disinfector 142.

As an embodiment, in a case in which the curtain 20 is made of woven fabric, the liquid-disinfectant-based disinfector 142, the ultraviolet-based disinfector 144, the plasma-based disinfector 148, and the heat-based disinfector 146 of the disinfection module 140 may be sequentially disposed.

As another embodiment, in a case in which the curtain 20 is made of synthetic resin, the liquid-disinfectant-based disinfector 142, the ultraviolet-based disinfector 144, and the plasma-based disinfector 148 of the disinfection module 140 may be sequentially disposed.

Therefore, as illustrated in FIG. 4B, the several disinfectors of the disinfection module 140 may apply plasma, heat, ultraviolet rays, a liquid disinfectant, and the like, which are required for the curtain 20 passing through the disinfection space, directly to the curtain 20, thereby disinfecting the curtain 20.

The movable curtain disinfection device 1 according to the present invention may further include a plurality of wheels 150 disposed on a lower portion of the support 10 and configured to move the frames 110 while supporting the frames 110.

The wheels 150 may be installed to be embedded in the lower portion of the support 10.

That is, the wheels 150 embedded in the lower portion of the support 10 may be extended and used to move the disinfection device, and the wheels 150 may be retracted into the support 10 to fix the disinfection device. Therefore, it is possible to reduce the inconvenience of having to separately collect and wash the curtain 20.

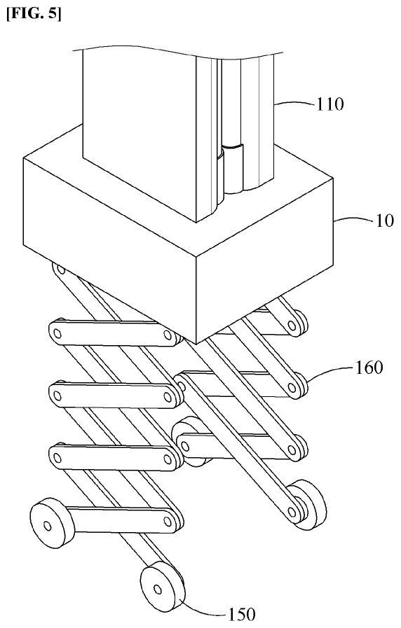

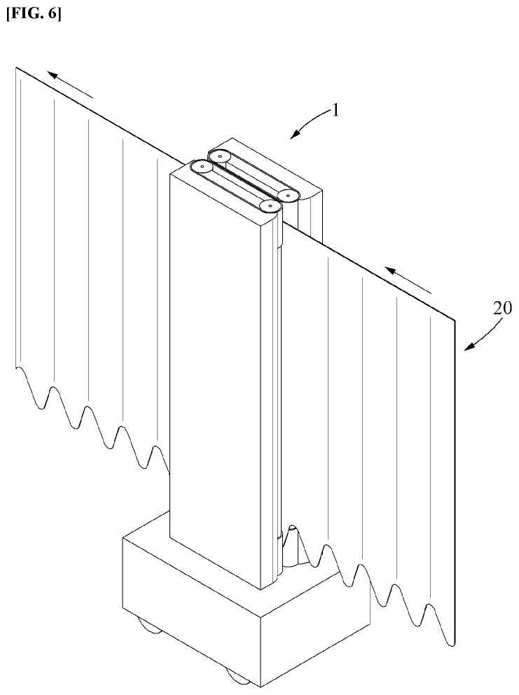

As illustrated in FIG. 5, the movable curtain disinfection device 1 according to the present invention may further include height adjustment members 160 capable of adjusting a height of the frame 110.

The height adjustment member 160 may be embedded in the support 10, connected between the lower portion of the support 10 and the wheels 150, and configured to adjust the height of the frame 110 in a vertical direction.

For example, the height adjustment member 160 may be implemented in the form of a lift jack or a hydraulic piston that performs a piston motion. Therefore, the height adjustment member 160 may be implemented in various forms.

Referring back to FIG. 1, the movable disinfection device for the curtain 20 according to the present invention may further include an operation panel 170 through which operation commands related to operations of the rotary roller 120, the rotary belt 130, and the disinfection module 140 are inputted.

The operation panel 170 may be disposed on an outer surface of the frame 110 so that a user may conveniently input the operation command. The operation panel 170 may be implemented as a touch-input or button-input type operation panel.

As an embodiment, the operation commands related to various functions such as rotational speeds of the rotary roller 120 and the rotary belt 130 and the amount of disinfection by the disinfection module 140 may be inputted to the operation panel 170.

As another embodiment, the operation panel 170 may output an image indicating a process of disinfecting the curtain 20 in the disinfection space.

Therefore, according to the embodiments of the present invention, the movable frames having the disinfection module using plasma, heat, ultraviolet ray, disinfectants, and the like may be implemented, which makes it possible to prevent infectious diseases by eliminating bacteria and viruses attached to the curtain in the hospital room.

In addition, according to the embodiments of the present invention, since the curtain disinfection device has the wheels, it is possible to reduce the inconvenience of having to separately collect and wash the curtains.

While the specific embodiments according to the present invention have been described above, various modifications may be made without departing from the scope of the present invention. Therefore, the scope of the present invention should not be limited to the described embodiments, and should be defined by not only the claims to be described below, but also those equivalent to the claims.

While the present invention has been described above with reference to the limited embodiments and the drawings, the present invention is not limited to the embodiments and may be variously modified and altered from the disclosure by those skilled in the art to which the present invention pertains. Therefore, the spirit of the present invention should be defined only by the appended claims, and all modifications, equivalents, and alternatives fall within the scope and spirit of the present invention.

1. A movable curtain disinfection device comprising:
frames installed on an upper portion of a support, spaced apart from each other to face each other, and configured to define a disinfection space for a curtain provided between the frames;
rotary rollers provided so that a pair of rotary rollers thereof is mounted on an inner surface of each of the frames, the rotary rollers being configured to rotate so that the curtain passes through the disinfection space;
rotary belts each configured to surround an outer portion of each of the pairs of rotary rollers, the rotary belts being configured to guide a movement of the curtain while rotating in conjunction with rotations of the rotary rollers; and
disinfection modules disposed between the pairs of rotary rollers and configured to disinfect the curtain passing through the disinfection space.

2. The movable curtain disinfection device of claim 1, wherein the rotary rollers comprise two pairs of rotary rollers mounted on the inner surfaces of the frames so as to face one another, and the rotary rollers are mounted in a longitudinal direction of the frame and disposed at two opposite sides of the inner surface of the frame.

3. The movable curtain disinfection device of claim 2, further comprising:
elastic members each disposed rearward of each of the pairs of rotary rollers and configured to press the rotary rollers in a direction in which the rotary rollers face one another so that the curtain passes through the disinfection space while being supported by the rotary rollers.

4. The movable curtain disinfection device of claim 1, wherein the rotary belts are provided at upper and lower sides of the rotary rollers.

5. The movable curtain disinfection device of claim 4, wherein the disinfection space is sealed as the rotary belts are in surface contact with the curtain passing through the disinfection space.

6. The movable curtain disinfection device of claim 1, wherein the disinfection module is a module detachably mounted on the inner surface of the frame and comprises at least one of a plasma-based disinfector, a heat-based disinfector, an ultraviolet-based disinfector, and a liquid-disinfectant-based disinfector.

7. The movable curtain disinfection device of claim 6, wherein the liquid-disinfectant-based disinfector, the ultraviolet-based disinfector, the plasma-based disinfector, and the heat-based disinfector of the disinfection module are sequentially disposed when the curtain is made of woven fabric, and
wherein the liquid-disinfectant-based disinfector, the ultraviolet-based disinfector, and the plasma-based disinfector are sequentially disposed when the curtain is made of synthetic resin.

8. The movable curtain disinfection device of claim 1, further comprising:
a plurality of wheels disposed on a lower portion of the support and configured to move the frame while supporting the frame.

9. The movable curtain disinfection device of claim 8, further comprising:
a height adjustment member provided to be embedded in the support, connected between the lower portion of the support and the wheels, and configured to adjust a height in a vertical direction.

10. The movable curtain disinfection device of claim 1, further comprising:
an operation panel disposed on an outer surface of the frame and configured such that operation commands related to operations of the rotary roller, the rotary belt, and the disinfection module are inputted into the operation panel.
